(19) United States
(12) Patent Application Publication
(10) Pub. No.: US 2012/0164634 A1
(43) Pub. Date: Jun. 28, 2012

(54) Method for Separating Particles and/or Cells Having 2 and More Surface Specificities

(75) Inventors: Hans-Werner Heinrich, Greifswald (DE); Jan-Michael Heinrich, Leipzig (DE)

(73) Assignee: Pluriselect GmbH, Leipzig (DE)

(21) Appl. No.: 13/390,408

(22) PCT Filed: Jul. 16, 2010
(86) PCT No.: PCT/DE2010/000830

§ 371 Date: Mar. 9, 2012

(30) Foreign Application Priority Data
Aug. 14, 2009 (DE) 10 2009 037 331.4

(51) Int. Cl. C12Q 1/68; G01N 33/566

(52) U.S. Cl. 435/6.1; 435/7.21

(57) ABSTRACT

The invention describes an easy method for the gentle separation of cells and/or particles from liquids, preferably blood. For that purpose known steps of magnetic separation procedures are combined with capture particle-assisted separating methods.

The invention relates to a method of separating particles and/or cells having two and more surface specificities. Fields of application of the invention are medicine and pharmaceutical chemistry.

### DESCRIPTION

Identifying and separating particles, in particular somatic cells and pathogens, from complex fluids such as blood is a necessary method step for carrying out research, diagnosis and treatment of diseases. Centrifugation methods and filters have proved useful for separating particles having different specific densities (for example leukocytes and erythrocytes). With the progress in research of the pathogenesis and immunogenesis of diseases, there is growing demand for identifying and separating cells having the same specific density but different functions. The different functions of lymphocytes, for example, are accompanied by expression of typical structures on the outer cell membrane (such as, for example, the “clusters of differentiation”—CDs). It is possible to generate antibodies to these structures, which are the crucial tool for the more specific separation methods. For decades, fluorescence-activated separation methods and magnetic separating methods have proved useful for this task, and Pluriselect (Germany) have recently started selling also a capture particle-assisted sieve separation method.

These methods allow to distinguish in a simple manner cells from complex fluids, which differ from other cells by a single feature on the membrane. Cells with different functions often display on their surface an identical feature (A), in addition to further features (B, C, . . . ) which are characteristic for other functions and are distributed differently within the cell population. The isolation of cells having only one desired combination of A, B or C is still a big challenge in biological research. Currently, this object can be achieved only by means of fluorescence-activated cell sorting (FACS) following multiple labelings.

FACS is without doubt the gold standard for separation of cells but is very demanding in terms of equipment and personnel. Other disadvantages of this technology are the extreme stress for the isolated cells, the complicated sterile techniques as well as methodical limits to the sorting of a relatively large number of vital of cells.

The problem is solved according to the invention by a novel combination of magnetic and capture particle-assisted separating methods. The invention is implemented according to claims 1 to 9.

### DESCRIPTION OF THE INVENTION

Exemplarily cells having the features A, B and C are to be isolated from blood. Said number of surface features results in seven different combinations (A, B, C, AB, AC, ABC, BC).

The problem is solved by using specific antibodies to A, B and C. In the present example, the antibodies to A are coupled to particles of 40 μm in diameter, antibodies to B are coupled to particles of 20 μm in diameter, and antibodies to C are coupled to magnetisable particles of <10 μm.

### Procedural Step 1:

The blood sample is then pre-incubated with detection systems A and C for 10 minutes. This is followed by adding detection system B and incubating for another 10 minutes. Subsequently, the sample is filtered through a sieve cascade with two sieves of mesh size 40 μm and 20 μm and rinsed adequately.

The 40 μm sieve retains: A, AB, AC, ABC

The 20 μm sieve retains: B and BC

In the flow-through: C

### Procedural Step 2:

All 3 samples are subjected to a magnetic separating method.

The 40 μm fraction is divided into: A, AB and AC, ABC.

The 20 μm fraction is separated into the homogeneous fractions B and BC.

From the flow-through, the homogeneous fraction C is separated.

### Procedural Step 3:

The fractions A, AB and AC, ABC are removed from capture particle A by known methods. A co-incubation with B is indicated. The samples are then separated via a 20 μm sieve.

The sieve retains: AB and ABC.

The flow-through contains A and AC.

### Method Step 4:

Both fractions are subjected to a magnetic separating method, resulting in separation of the homogeneous fractions AC and ABC, and A and AB.

The method can be carried out using large numbers of cells, with little effort in a short time and with low stress for the cells. A person skilled in the art may develop wide combinations by combining magnetic and size-defined capture particles in a suitable manner.

1. A method of separating particles or cells having two or more surface specificities, comprising a combination of particle-assisted and magnetic separations, wherein the mixture containing particles or cells is incubated successively or simultaneously
with capture particles for surface specificity A,
with capture particles for surface specificity B, and,
where appropriate, with capture particles for surface specificity C,
and, where appropriate for further specificities,
wherein the capture particles have different sizes and/or are magnetisable, are subsequently separated by means of sieve membranes or subjected to a magnetic separating method, and are then treated further, where appropriate.

2. Method according to claim 1, further comprising magnetisable capture particles that are smaller than the non-magnetisable capture particles.

3. Method according to claim 1, further comprising that the non-magnetisable capture particles are separated by means of sieve membranes.

4. Method according to claim 3, further comprising a plurality of non-magnetisable capture particles that are separated by means of sieves/membranes having the mesh/pore size required therefor.

5. Method according to claim 1, further comprising that the non-magnetisable capture particles are separated by means of magnetic field action.

6. Method according to claim 1, further comprising nucleic acids, peptides or proteins, preferably antibodies, that are used as capture specificities.

7. Method according to claim 1, further comprising that the fixed particles/cells are removed by methods known per se.

8. Use of the method according to claim 1 for separating particles and/or cells from complex fluids, preferably blood, for research purposes and for the diagnosis and therapy of diseases.

9. (canceled)
